(19) United States
(12) Patent Application Publication
(10) Pub. No.: US 2005/0149091 A1
(43) Pub. Date: Jul. 7, 2005

(54) GUIDED OSTEOTOMES

(76) Inventors: Linggawati Tanamal, Fort Wayne, IN (US); James C. Harris, Warsaw, IN (US); Kim C. Bertin, Bountiful, UT (US); William R. Kennedy, Sarasota, FL (US)

Correspondence Address: ZIMMER TECHNOLOGY - ROBERTS;INDIANO, VAUGHN & ROBERTS, 1 NORTH PENNSYLVANIA AVENUE #850, INDIANAPOLIS, IN, 46204, US

(21) Appl. No.: 10/751,125

(22) Filed: Jan. 3, 2004

(51) Int. Cl. A61B 17/32

(52) U.S. Cl. 606/184

(57) ABSTRACT

An osteotome including a head portion is provided. The head portion includes a first substantially planar surface and a second substantially planar surface extending obtusely from the first substantially planar surface. The osteotome further includes a bone cutter extending from the first substantially planar surface, and an elongated portion extending from the head portion. The first substantially planar surface and the second substantially planar surface face away from the elongated portion.

### FIELD OF THE INVENTION

The present invention relates generally to the field of orthopaedics, and, more particularly, to osteotomes.

### BACKGROUND

An osteoma is a benign tumor composed of bone tissue. Osteomas can arise without obvious cause. An osteophyte (sometimes called a “bone spur”) is a projection of bone tissue that can form on a bone of a joint when an animal's body attempts to better distribute weight across a surface that has been damaged by arthritis or other conditions. Osteomas and osteophytes can limit joint motion and contribute to joint pain, and they can become increasingly more restrictive and painful as they grow.

Historical approaches to the removal of osteomas and osteophytes from hard to reach areas have required tedious manipulations of specialized cutting tools, or osteotomes. FIG. 1 (prior art) shows a lateral view of a simplified prosthetic knee 10 (including a distal femoral component 20, a meniscial component 30, and a proximal tibial component 40); and FIG. 2 (prior art) shows a historical removal of an osteoma or osteophyte 50 from a posterior condylar region 60 with a simplified conventional osteotome 70 in preparation of a distal femur 80 for receiving the distal femoral component 20 of FIG. 1. After end portions of the distal femur 80 and a corresponding proximal tibia 90 (see FIG. 2) have been removed and the remaining surfaces have been planed or otherwise reshaped to receive the prosthetic knee 10 (see FIG. 1), the historical approach has required hyperflexion of the knee joint to gain access to the osteoma or osteophyte 50 with the osteotome 70 (see FIG. 2), followed by manipulation of the osteotome 70 to remove the osteoma or osteophyte 50.

Such extreme flexions of the joints have been difficult to achieve for patients with large extremities and/or other physical limitations, and manipulations and guidance of conventional osteotomes have been undesirably tedious and time consuming.

### SUMMARY OF THE INVENTION

The present invention provides an osteotome including a head portion. The head portion includes a first substantially planar surface and a second substantially planar surface extending obtusely from the first substantially planar surface. The osteotome further includes a bone cutter extending from the first substantially planar surface, and an elongated portion extending from the head portion. The first substantially planar surface and the second substantially planar surface face away from the elongated portion.

In an alternative embodiment, the present invention provides an osteotome equipped to guide a bone cutter relative to a bone surface and to transmit at least a portion of an externally generated impact force to the bone cutter. The osteotome includes means for guiding the bone cutter relative to the bone surface and means, coupled to the means for guiding, for receiving the impact force.

In another alternative embodiment, the present invention provides an osteotome capable of being guided against bone surfaces. The osteotome includes a head portion. The head portion includes a first substantially planar surface and a second substantially planar surface extending from the first substantially planar surface at an angle of about 95 degrees. The osteotome further includes a single-edged bone cutter extending generally perpendicularly from the first substantially planar surface, a rod extending from the head portion, a handle coupled to the rod, and an anvil abutting the handle. The first substantially planar surface and the second substantially planar surface face away from the rod, and the first substantially planar surface and the second substantially planar surface are smooth enough to slide against the bone surfaces without significantly abrading the bone surfaces.

In another alternative embodiment, the present invention provides an osteotome capable of being guided against bone surfaces. The osteotome includes a head portion including an anvil, a first substantially planar surface, and a second substantially planar surface extending from the first substantially planar surface at an angle of one of about 95 and 135 degrees. The osteotome further includes a single-edged bone cutter extending generally perpendicularly from the first substantially planar surface, a rod extending from the head portion, and a handle coupled to the rod. The first substantially planar surface and the second substantially planar surface face away from the rod, and the first substantially planar surface and the second substantially planar surface are smooth enough to slide against the bone surfaces without significantly abrading the bone surfaces.

The above-noted features and advantages of the present invention, as well as additional features and advantages, will be readily apparent to those skilled in the art upon reference to the following detailed description and the accompanying drawings.

### BRIEF DESCRIPTION OF THE DRAWINGS

FIG. 1 (prior art) shows a lateral view of a simplified prosthetic knee;

FIG. 2 (prior art) shows a historical removal of an osteoma or osteophyte from a posterior condylar region with a simplified conventional osteotome in preparation of a distal femur for receiving the distal femoral component of FIG. 1;

FIG. 3 shows a lateral view of an exemplary osteotome according to the present invention;

FIG. 4 shows a perspective view of the exemplary osteotome of FIG. 3;

FIG. 5 shows a lateral view of an exemplary alternative osteotome according to the present invention;

FIG. 6 shows a perspective view of the exemplary alternative osteotome of FIG. 5;

FIG. 7 shows a lateral view of exemplary operations of the exemplary osteotome of FIG. 3 for an exemplary removal of an exemplary posterior condylar osteophyte from an exemplary knee joint;

FIG. 8 shows a lateral view of exemplary operations of the exemplary osteotome of FIG. 5 for an exemplary removal of the exemplary osteophyte of FIG. 7;

FIG. 9 shows a lateral view of another exemplary alternative osteotome according to the present invention;

FIG. 10 shows a perspective view of the exemplary osteotome of FIG. 9; and

FIG. 11 shows a lateral view of exemplary operations of the exemplary osteotome of FIG. 9 for an exemplary removal of the exemplary osteophyte of FIG. 7.

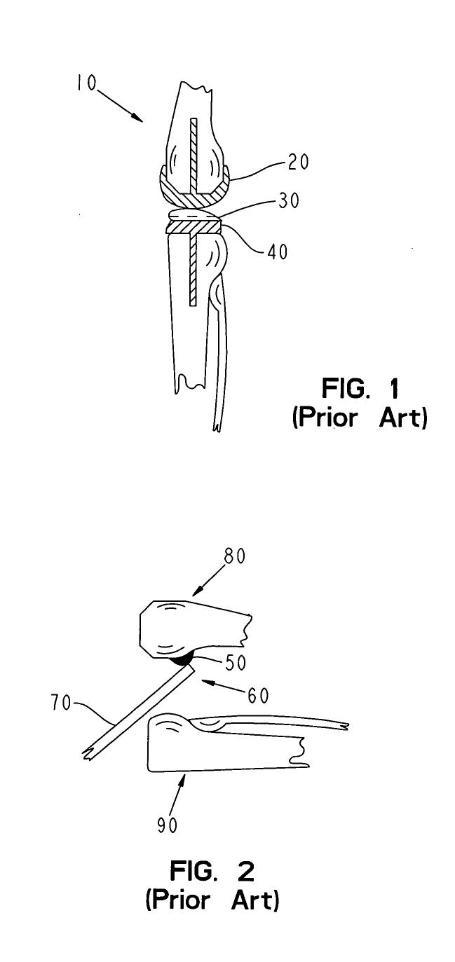

### DETAILED DESCRIPTION OF EXEMPLARY EMBODIMENT(S)

Like reference numerals indicate like parts throughout this description and the accompanying drawings.

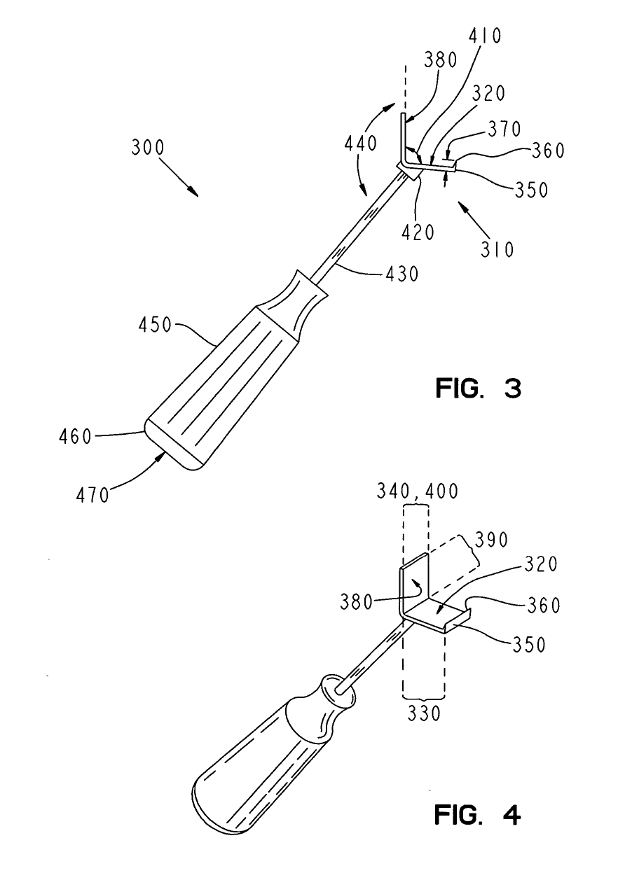

FIG. 3 shows a lateral view of an exemplary osteotome 300 according to the present invention. The various components of osteotome 300 are made from stainless steel, plastic, or any other material(s) suitable for use in surgical procedures. Additionally, in the exemplary embodiment osteotome 300 is reusable and, accordingly, is made suitable for sterilization in an autoclave. In alternative embodiments, one or more components (or all) of osteotome 300 may be disposable.

Osteotome 300 includes a head portion 310. Portion 310 includes a substantially planar surface 320. In the exemplary embodiment, surface 320 has a length 330 of about 0.75 inches (see FIG. 4) and a width 340, transverse to length 330, of about 0.5 inches (see FIG. 4). It is noted, however, that in alternative embodiments length 330 and/or width 340 may be arbitrarily increased or decreased to provide practically any number of preset sizes for portion 310, or they may be tailored to fit a particular patient based on predetermined imaging data or any other specifications. Surface 320 is smooth enough to slide against bone surfaces without significantly abrading them.

Portion 310 further includes a bone cutter 350 that extends generally perpendicularly from surface 320 and spans width 340 (see FIG. 4). Cutter 350 is configured to cut bone and, more particularly, to shave or scrape away an osteoma or osteophyte. In the exemplary embodiment, cutter 350 tapers as it extends from surface 320 to define a single sharp edge 360 positioned at a height 370 (about 0.2 inches) above surface 320. It is noted, however, that in alternative embodiments height 370 may be arbitrarily increased or decreased to provide practically any number of preset sizes for cutter 350, or it may be tailored to fit a particular patient based on predetermined imaging data or any other specifications. Moreover, in alternative embodiments cutter 350 may be a burr or rasp, may otherwise be multi-facetted or multi-edged, may be of any other suitable size and/or configuration, and/or may extend from surface 320 in any other suitable direction or orientation relative to surface 320.

Portion 310 also includes a substantially planar surface 380. Surface 380 has a height 390 of about 0.75 inches (see FIG. 4) and a width 400, transverse to height 390, of about 0.5 inches (see FIG. 4). It is noted, however, that in alternative embodiments height 390 and/or width 400 may be arbitrarily increased or decreased to provide practically any number of preset sizes for surface 380, or they may be tailored to fit a particular patient based on predetermined imaging data or any other specifications. Surface 380 extends from surface 320 at an obtuse angle 410. In the exemplary embodiment, angle 410 is about 95 degrees. Surface 380 is smooth enough to slide against bone surfaces without significantly abrading them.

Portion 310 further includes a suitable connecting block or mass 420. Mass 420 is positioned outside of angle 410, generally centered relative to width 340 (and width 400), and generally proximal to the vertex of angle 410 (but offset towards cutter 350) relative to length 330.

Osteotome 300 further includes an elongated portion 430 that extends from mass 420 at an angle 440 relative to surface 380. In the exemplary embodiment, portion 430 is a shaft or rod and angle 440 is about 130 degrees. Accordingly, it should be appreciated that in the exemplary embodiment surface 320 and surface 380 both face away from elongated portion 430. In alternative embodiments, elongated portion 430 may be narrower, wider, may include additional curvatures, angles and/or contours, or may be configured in any other suitable manner, and angle 440 may be any other suitable number of degrees.

Osteotome 300 also includes a handle 450. Handle 450 extends from elongated portion 430.

Osteotome 300 further includes an anvil 460 that abuts handle 450. Anvil 460 includes a substantially planar surface 470. Anvil 460 is configured to receive strikes from a user's hammer or mallet onto surface 470 during operation.

FIG. 4 shows a perspective view of the exemplary osteotome 300 of FIG. 3. Surface 320, length 330, width 340 (and width 400), cutter 350, edge 360, surface 380, and height 390 are discernable in FIG. 4.

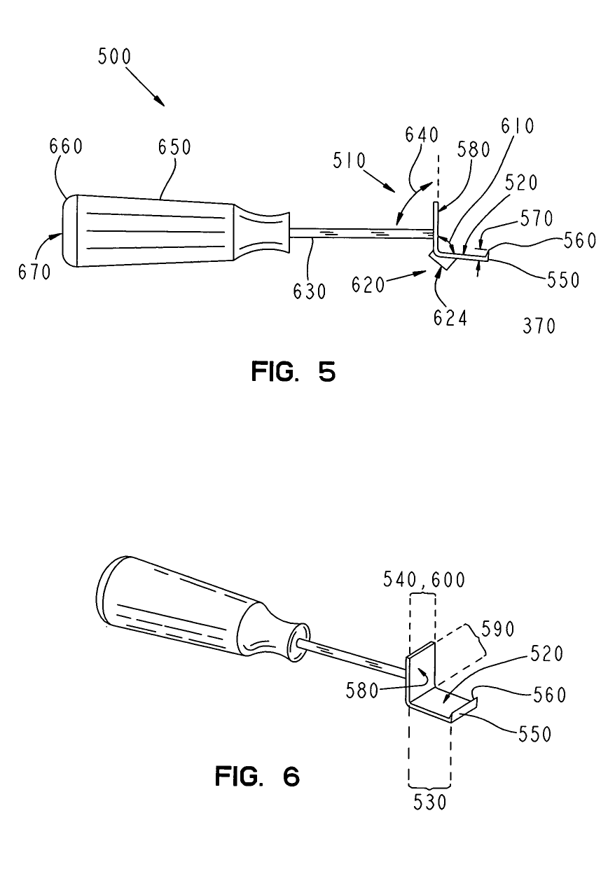

FIG. 5 shows a lateral view of an exemplary alternative osteotome 500 according to the present invention. The various components of osteotome 500 are made from stainless steel, plastic, or any other material(s) suitable for use in surgical procedures. Additionally, in the exemplary embodiment osteotome 500 is reusable and, accordingly, is made suitable for sterilization in an autoclave. In alternative embodiments, one or more components (or all) of osteotome 500 may be disposable.

Osteotome 500 includes a head portion 510. Portion 510 includes a substantially planar surface 520. Surface 520 has a length 530 of about 0.75 inches (see FIG. 6) and a width 540, transverse to length 530, of about 0.5 inches (see FIG. 6). It is noted, however, that in alternative embodiments length 530 and/or width 540 may be arbitrarily increased or decreased to provide practically any number of preset sizes for surface 520, or they may be tailored to fit a particular patient based on predetermined imaging data or any other specifications. Surface 520 is smooth enough to slide against bone surfaces without significantly abrading them.

Portion 510 further includes a bone cutter 550 that extends generally perpendicularly from surface 520 and spans width 540 (see FIG. 6). Cutter 550 is configured to cut bone and, more particularly, to shave or scrape away an osteoma or osteophyte. In the exemplary embodiment, cutter 550 tapers as it extends from surface 520 to define a single sharp edge 560 positioned at a height 570 (about 0.2 inches) above surface 520. It is noted, however, that in alternative embodiments height 570 may be arbitrarily increased or decreased to provide practically any number of preset sizes for cutter 550, or it may be tailored to fit a particular patient based on predetermined imaging data or any other specifications. Moreover, in alternative embodiments cutter 550 may be a burr or rasp, may otherwise be multi-facetted or multi-edged, may be of any other suitable size and/or configuration, and/or may extend from surface 520 in any other suitable direction or orientation relative to surface 520.

Portion 510 also includes a substantially planar surface 580. Surface 580 has a height 590 of about 0.75 inches (see FIG. 6) and a width 600, transverse to height 590, of about 0.5 inches (see FIG. 6). It is noted, however, that in alternative embodiments height 590 and/or width 600 may be arbitrarily increased or decreased to provide practically any number of preset sizes for surface 580, or they may be tailored to fit a particular patient based on predetermined imaging data or any other specifications. Surface 580 extends from surface 520 at an obtuse angle 610. In the exemplary embodiment, angle 610 is about 95 degrees. Surface 580 is smooth enough to slide against bone surfaces without significantly abrading them.

Portion 510 further includes an anvil 620. Anvil 620 is positioned outside of angle 610, generally centered relative to width 540 (and width 600), and generally proximal to the vertex of angle 610 (but offset towards cutter 550) relative to length 530. Anvil 620 includes a substantially planar surface 624. Anvil 620 is configured to receive strikes from a user's hammer or mallet onto surface 624 during operation.

Osteotome 500 further includes an elongated portion 630 that extends from portion 510 at an angle 640 relative to surface 580. In the exemplary embodiment, portion 630 is a shaft or rod and angle 640 is about 90 degrees. Accordingly, it should be appreciated that in the exemplary embodiment surface 520 and surface 580 both face away from elongated portion 630. In alternative embodiments, elongated portion 630 may be narrower, wider, may include additional curvatures, angles and/or contours, or may be configured in any other suitable manner, and angle 640 may be any other suitable number of degrees.

Osteotome 500 also includes a handle 650. Handle 650 extends from elongated portion 630.

Osteotome 500 further includes an anvil 660 that abuts handle 650. Anvil 660 includes a substantially planar surface 670. Anvil 660 is configured to receive strikes from a user's hammer or mallet onto surface 670 during operation.

FIG. 6 shows a perspective view of the exemplary alternative osteotome 500 of FIG. 5. Surface 520, length 530, width 540 (and width 600), cutter 550, edge 560, surface 580, and height 590 are discernable in FIG. 6.

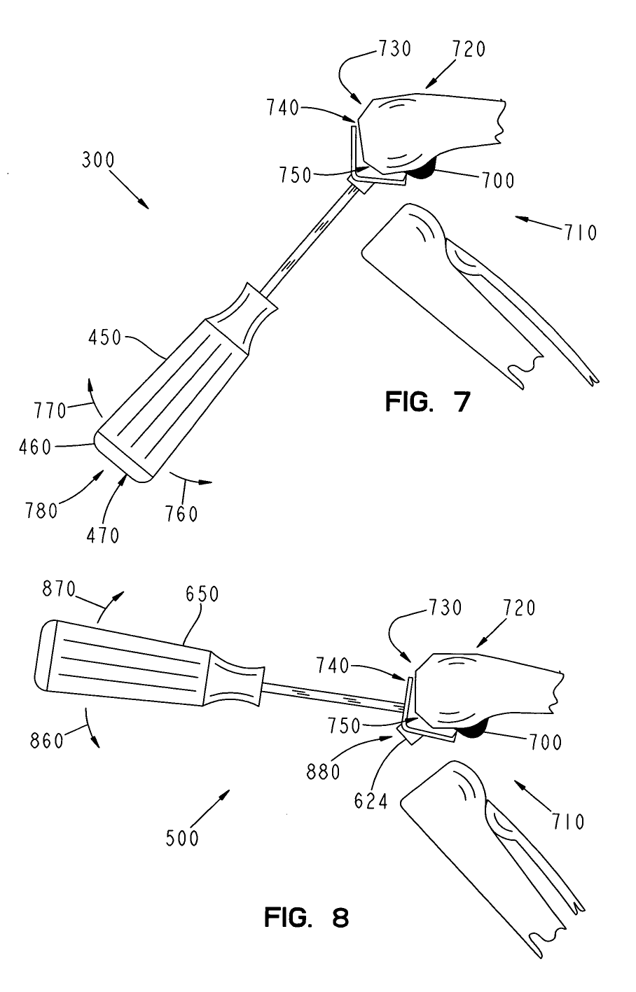

FIG. 7 shows a lateral view of exemplary operations of the exemplary osteotome 300 of FIG. 3 for an exemplary removal of an exemplary posterior condylar osteophyte 700 from an exemplary knee joint 710. For removal of the posterior condylar osteophyte 700, a surgeon or other user makes a suitable incision(s) and/or otherwise creates a suitable pathway(s) and a working space(s) in and around joint 710 in a known manner. This may include everting (i.e., “flipping aside”) the corresponding patella (not shown) or any other suitable procedures. A corresponding distal femur 720 is resected in a known manner to provide an anterior distal femoral cut surface 730, a distal distal femoral cut surface 740, and a posterior distal femoral cut surface 750. It is noted, however, that alternative embodiments of the present invention may be used to remove osteomas and/or osteophytes from the hip, the elbow, or other areas, and the procedures to gain access to such osteomas and/or osteophytes and to resect the corresponding bone surfaces may vary accordingly.

Continuing the example of FIG. 7, portion 310 (see FIG. 3 and FIG. 4) is inserted into joint 710 (from the anterior) such that surface 380 of portion 310 (see FIG. 3 and FIG. 4) abuts surface 740 and edge 360 of cutter 350 (see FIG. 3 and FIG. 4) is proximal to (or even abuts) osteophyte 700. While maintaining the abutment of surface 380 of portion 310 (see FIG. 3 and FIG. 4) against surface 740 of distal femur 720, handle 450 is generally alternately manipulated (or, in other words, rocked) along directional line 760 and directional line 770, such that surface 380 (see FIG. 3 and FIG. 4) slides against surface 740 as cutter 350 (see FIG. 3 and FIG. 4) works to remove osteophyte 700. To force and/or facilitate movement of handle 450 along directional line 760 (and thus, removal of osteophyte 700 by cutter 350), an external force is applied by striking surface 470 of anvil 460 generally along directional line 780 with a hammer, mallet, or any other suitable instrument.

Operations of osteotome 300 are suitably continued until osteophyte 700 is suitably removed. A visual determination that osteophyte 700 has been suitably removed may be made with a naked eye or with the aid of a remote camera or other suitable imaging device. Alternatively or additionally, a tactile determination that osteophyte 700 has been suitably removed may be made by alternately operating osteotome 300 and feeling the corresponding area of distal femur 720 with a finger. Moreover, the determination that osteophyte 700 has been suitably removed may be made in any other suitable manner. Accordingly, it is noted that suitable embodiments of the present invention may be coupled to a haptic arm, a robotic assembly, and/or computerized device, or the like, for an automatic determination that osteophyte 700 has been suitably removed.

FIG. 8 shows a lateral view of exemplary operations of the exemplary osteotome 500 of FIG. 5 for an exemplary removal of the exemplary osteophyte 700 of FIG. 7. For removal of osteophyte 700 with osteotome 500, a surgeon or other user makes a suitable incision(s) and/or otherwise creates a suitable pathway(s) and a working space(s) in and around joint 710 in a known manner. This may include everting (i.e., “flipping aside”) the corresponding patella (not shown) or any other suitable procedures. A corresponding distal femur 720 is resected in a known manner to provide an anterior distal femoral cut surface 730, a distal distal femoral cut surface 740, and a posterior distal femoral cut surface 750. It is noted, however, that alternative embodiments of the present invention may be used to remove osteomas and/or osteophytes from the hip, the elbow, or other areas, and the procedures to gain access to such osteomas and/or osteophytes and to resect the corresponding bone surfaces may vary accordingly.

Continuing the example of FIG. 8, portion 510 (see FIG. 5 and FIG. 6) is inserted into joint 710 (from the anterior) such that surface 580 of portion 510 (see FIG. 5 and FIG. 6) abuts surface 740 and edge 560 of cutter 550 (see FIG. 5 and FIG. 6) is proximal to (or even abuts) osteophyte 700. While maintaining the abutment of surface 580 of portion 510 (see FIG. 5 and FIG. 6) against surface 740 of distal femur 720, handle 650 is generally alternately manipulated (or, in other words, rocked) along directional line 860 and directional line 870, such that surface 580 (see FIG. 5 and FIG. 6) slides against surface 740 as cutter 550 (see FIG. 5 and FIG. 6) works to remove osteophyte 700. To force and/or facilitate movement of handle 650 along directional line 760 (and thus, removal of osteophyte 700 by cutter 550), an external force is applied by striking surface 624 of anvil 620 (see FIG. 5) generally along directional line 880 with a hammer, mallet, or any other suitable instrument.

Operations of osteotome 500 are suitably continued until osteophyte 700 is suitably removed. A visual determination that osteophyte 700 has been suitably removed may be made with a naked eye or with the aid of a remote camera or other suitable imaging device. Alternatively or additionally, a tactile determination that osteophyte 700 has been suitably removed may be made by alternately operating osteotome 500 and feeling the corresponding area of distal femur 720 with a finger. Moreover, the determination that osteophyte 700 has been suitably removed may be made in any other suitable manner. Accordingly, it is noted that suitable embodiments of the present invention may be coupled to a haptic arm, a robotic assembly, and/or computerized device, or the like, for an automatic determination that osteophyte 700 has been suitably removed.

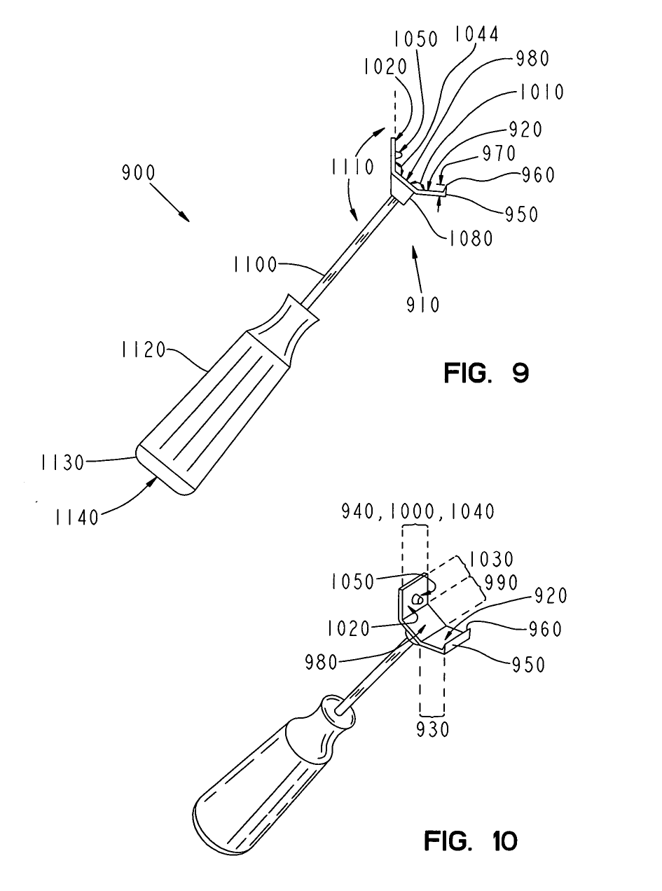

FIG. 9 shows a lateral view of another exemplary alternative osteotome 900 according to the present invention. The various components of osteotome 900 are made from stainless steel, plastic, or any other material(s) suitable for use in surgical procedures. Additionally, in the exemplary embodiment osteotome 900 is reusable and, accordingly, is made suitable for sterilization in an autoclave. In alternative embodiments, one or more components (or all) of osteotome 900 may be disposable.

Osteotome 900 includes a head portion 910. Portion 910 includes a substantially planar surface 920. In the exemplary embodiment, surface 920 has a length 930 of about 0.5 inches (see FIG. 10) and a width 940, transverse to length 930, of about 0.5 inches (see FIG. 10). It is noted, however, that in alternative embodiments length 930 and/or width 940 may be arbitrarily increased or decreased to provide practically any number of preset sizes for portion 910, or they may be tailored to fit a particular patient based on predetermined imaging data or any other specifications. Surface 920 is smooth enough to slide against bone surfaces without significantly abrading them.

Portion 910 further includes a bone cutter 950 that extends generally perpendicularly from surface 920 and spans width 940 (see FIG. 10). Cutter 950 is configured to cut bone and, more particularly, to shave or scrape away an osteoma or osteophyte. In the exemplary embodiment, cutter 950 tapers as it extends from surface 920 to define a single sharp edge 960 positioned at a height 970 (about 0.2 inches) above surface 920. It is noted, however, that in alternative embodiments height 970 may be arbitrarily increased or decreased to provide practically any number of preset sizes for cutter 950, or it may be tailored to fit a particular patient based on predetermined imaging data or any other specifications. Moreover, in alternative embodiments cutter 950 may be a burr or rasp, may otherwise be multi-facetted or multi-edged, may be of any other suitable size and/or configuration, and/or may extend from surface 920 in any other suitable direction or orientation relative to surface 920.

Portion 910 also includes a substantially planar surface 980. Surface 980 has a height 990 of about 0.5 inches (see FIG. 10) and a width 1000, transverse to height 990, of about 0.5 inches (see FIG. 10). It is noted, however, that in alternative embodiments height 990 and/or width 1000 may be arbitrarily increased or decreased to provide practically any number of preset sizes for surface 980, or they may be tailored to fit a particular patient based on predetermined imaging data or any other specifications. Surface 980 extends from surface 920 at an obtuse angle 1010. In the exemplary embodiment, angle 1010 is about 135 degrees. Surface 980 is smooth enough to slide against bone surfaces without significantly abrading them.

Portion 910 further includes a substantially planar surface 1020. In the exemplary embodiment, surface 1020 has a height 1030 of about 0.4 inches (see FIG. 10) and a width 1040, transverse to length 1030, of about 0.5 inches (see FIG. 10). It is noted, however, that in alternative embodiments height 1030 and/or width 1040 may be arbitrarily increased or decreased to provide practically any number of preset sizes for portion 910, or they may be tailored to fit a particular patient based on predetermined imaging data or any other specifications. Surface 1020 extends from surface 980 at an obtuse angle 1044. In the exemplary embodiment, angle 1044 is about 135 degrees. Surface 1020 is smooth enough to slide against bone surfaces without significantly abrading them.

Portion 910 further includes a generally mogul-shaped protuberance 1050 that extends generally perpendicularly from surface 1020 and is roughly centered on surface 1020 (see FIG. 10). In the exemplary embodiment, protuberance 1050 extends to an apex about 0.2 inches from the plain of surface 1020. It is noted, however, that in alternative embodiments the size of protuberance 1050 may be arbitrarily increased or decreased to any number of preset sizes, or it may be tailored to fit a particular patient based on predetermined imaging data or any other specifications. Moreover, in alternative embodiments protuberance 1050 may be a ridge that spans width 1040, may be of any other suitable size and/or configuration, and/or may extend from surface 1020 in any other suitable direction or orientation relative to surface 1020.

Portion 910 further includes a suitable connecting block or mass 1080. Mass 1080 is positioned outside of angle 1010 and outside of angle 1044, generally centered relative to width 940 (and width 1000, and width 1040), and generally proximal to the vertex of angle 1010 (but offset towards angle 1044) relative to height 990.

Osteotome 900 further includes an elongated portion 1100 that extends from mass 1080 at an angle 1110 relative to surface 1020. In the exemplary embodiment, portion 1100 is a shaft or rod and angle 1110 is about 135 degrees. Accordingly, it should be appreciated that in the exemplary embodiment surface 920, surface 980, and surface 1020 all face away from elongated portion 1100. In alternative embodiments, elongated portion 1100 may be narrower, wider, may include additional curvatures, angles and/or contours, or may be configured in any other suitable manner, and angle 1110 may be any other suitable number of degrees.

Osteotome 900 also includes a handle 1120. Handle 1120 extends from elongated portion 1100.

Osteotome 900 further includes an anvil 1130 that abuts handle 1120. Anvil 1130 includes a substantially planar surface 1140. Anvil 1130 is configured to receive strikes from a user's hammer or mallet onto surface 1140 during operation.

FIG. 10 shows a perspective view of the exemplary osteotome 900 of FIG. 9. Surface 920, length 930, width 940 (and width 1000, and width 1040), cutter 950, edge 960, surface 980, height 990, surface 1020, height 1030, and protuberance 1050 are discernable in FIG. 10.

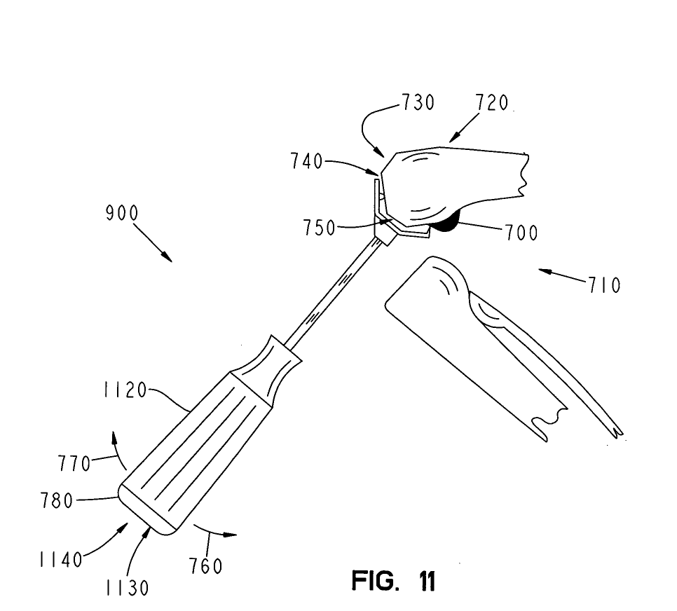

FIG. 11 shows a lateral view of exemplary operations of the exemplary osteotome 900 of FIG. 9 for an exemplary removal of the exemplary osteophyte 700 of FIG. 7. It should be readily appreciated that operations of exemplary osteotome 900 are like those of exemplary osteotome 300 (see FIG. 3 and FIG. 7), except that for operations of osteotome 900 protuberance 1050 (see FIG. 9 and FIG. 10) abuts and slides against surface 740; whereas for operation of osteotome 300 surface 380 abuts and slides against surface 740.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. An osteotome, comprising:
a head portion including a first substantially planar surface, and a second substantially planar surface extending at an obtuse angle from said first substantially planar surface;
a bone cutter extending and generally perpendicular from the first substantially planar surface, said bone cutter tapering as it extends away from said first substantially planar surface; and
an elongated portion extending from said head portion;
wherein said first substantially planar surface and said second substantially planar surface face away from said elongated portion.

2. The osteotome of claim 1, further including a handle coupled to the elongated portion.

3. The osteotome of claim 2, further including an anvil coupled to the handle.

4. The osteotome of claim 1, wherein the head portion further includes an anvil.

5. The osteotome of claim 1, wherein said head portion further includes a third substantially planar surface extending at an obtuse angle from said second substantially planar surface.

6. The osteotome of claim 5, wherein said second planar surface has a width and a length and said head portion further includes a protuberance extending from the second substantially planar surface at generally the midpoint of the width and length of said second planar surface.

7. The osteotome of claim 6, wherein the protuberance is generally mogul-shaped.

8. The osteotome of claim 1, wherein the second substantially planar surface extends from the first substantially planar surface at an angle of about 95 degrees.

9. The osteotome of claim 5, wherein the second substantially planar surface extends from the first substantially planar surface at an angle of about 135 degrees and the third substantially planar surface extends from the second substantially planar surface at an angle of about 135 degrees.

10. The osteotome of claim 1, wherein the bone cutter extends generally perpendicularly from the first substantially planar surface.

11. The osteotome of claim 10, wherein the bone cutter defines a single sharp edge.

12. The osteotome of claim 1, wherein the elongated portion defines a rod.

13. (canceled)

14. (canceled)

15. An osteotome capable of being guided against bone surfaces, the osteotome comprising:
a head portion including a first substantially planar surface, and a second substantially planar surface extending from said first substantially planar surface at an angle of about 95 degrees;
a bone cutter extending upward and generally perpendicularly from said first substantially planar surface, said bone cutter tapering to a single cutting edge as it extends away from said first substantially planar surface;
a rod extending from said head portion;
a handle coupled to said rod; and
an anvil abutting said handle;
wherein said first substantially planar surface and said second substantially planar surface face away from said rod, and said first substantially planar surface and said second substantially planar surface are smooth enough to slide against the bone surfaces without significantly abrading the bone surfaces.

16. An osteotome capable of being guided against bone surfaces, the osteotome comprising:
a head portion including an anvil, a first substantially planar surface, and a second substantially planar surface extending from said first substantially planar surface at an angle of one of about 95 and 135 degrees;
a bone cutter extending upward and generally perpendicularly from said first substantially planar surface, said bone cutting tapering to a single cutting edge as it extends upward from said first substantially planar surface;
a rod extending from said head portion; and
a handle coupled to said rod;
wherein said first substantially planar surface and said second substantially planar surface face away from said rod, said first substantially planar surface and said second substantially planar surface are smooth enough to slide against the bone surfaces without significantly abrading the bone surfaces.
